(19) United States
(12) Patent Application Publication
(10) Pub. No.: US 2019/0160095 A1
(43) Pub. Date: May 30, 2019

(54) METHODS OF DETERRING CANINE COPROPHAGY

(71) Applicant: The Regents of the University of California, Oakland, CA (US)

(72) Inventor: Benjamin L. HART, Davis, CA (US)

(21) Appl. No.: 16/202,669

(22) Filed: Nov. 28, 2018

Related U.S. Application Data
(60) Provisional application No. 62/592,413, filed on Nov. 30, 2017.

(51) Int. Cl. A61K 33/14; A61K 35/37; A61P 1/00

(52) U.S. Cl. CPC A61K 33/14; A61P 1/00; A61K 35/37

(57) ABSTRACT

Provided are compositions, devices and methods for deterring coprophagy in canines.

### CROSS-REFERENCE TO RELATED APPLICATIONS

This application claims the benefit of U.S. Provisional Patent Application No. 62/592,413 filed Nov. 30, 2017, which application is incorporated herein by reference in its entirety.

### BACKGROUND

A puzzling, but common, behavior in some domestic dogs is a persistent tendency to consume their own feces or those of other adult dogs. This problem, occurring in an estimated 16 percent of domestic dogs (see study below), is very upsetting to the dog owners and can even lead to relinquishment of the dog. While there seems to be no clinically established abnormality associated with the behavior, such as a gastrointestinal upset, nutritional deficiency or compulsive disorder, dog owners are often very disturbed by the behavior. In fact, currently, there are 11 commercial products specifically marketed for dealing with the problem: 21st Century Deterrence®; Coproban®; Deter®; Dis-Taste®; For-Bid®; Nasty Habit®; NaturVet Deterrent®; Potty Mouth®; S.E.P®; Stop Stool Eating®; Stop Tablets®.

A rather interesting paradox, presented by the occurrence of conspecific coprophagy, is that dogs seem to find conspecific feces aversive and typically keep their rest areas clean by eliminating outside the house (1). This aversion to feces is viewed as an innate behavioral adaptation inherited from wild wolf ancestors for avoiding exposure to fecal-borne intestinal parasites and pathogens (2,3). In nature, wolf and other canid feces typically carry intestinal parasites as identified in scats (4-8).

There are no data-based published studies dealing with the overall prevalence of conspecific coprophagy in domestic dogs or demographic factors, such as association with breed, gender, age, number of dogs in the household, diet or eating style. And, there are no data on the evaluation of efficacy of commercial products marketed specifically for the syndrome or the use of behavior modification approaches to eliminate the problem long-term.

One study reported that 28 percent of the dogs surveyed engaged in eating of either herbivore or canine stools, but did not distinguish between the two behaviors (9). A study of 14 coprophagic Labrador Retrievers found that punishing of attempts at eating stools with a citronella spray reduced the behavior by about two-thirds during the three-week trial, but the long-term success was not reported (10).

The topic of canine coprophagy was addressed at UC Davis with four objectives. One was to collect demographic data on the prevalence of conspecific stool eating by dogs in the general population and examine demographic factors such as gender, spaying or neutering, age, number of dogs in the household, type of food eaten, eating behavior style and breed of the dog. The second objective was to look at the association of coprophagy with aversion, or absence of aversion, to conspecific feces as indicated by ease or difficulty in house training. A third objective was to establish the characteristics of stool eating especially with regard to age of dog stools eaten. A fourth objective was to evaluate the therapeutic success of various behavior modification approaches and the use of commercial products specifically marketed for treating stool eating. It was envisioned that learning about conspecific coprophagy in dogs might reveal some useful information in understanding and dealing with this problem behavior.

Two contrasting testable hypotheses were considered. One is that coprophagic dogs are exhibiting an abnormal behavior stemming from one or more contributing causes such as weak aversion to feces, a dietary deficiency, and/or association with a recognized compulsive behavior. Depending on the possible cause, the predictions of this hypothesis were that: coprophagic dogs would be more difficult to housetrain than non-coprophagic dogs, reflecting poor feces aversion; coprophagic dogs would be fed a diet markedly different than that of non-coprophagic dogs; and/or coprophagic dogs would be more likely than non-coprophagic dogs to show one or more compulsive behaviors, such as tail chasing. Based on a presumed motivation for commercial production of food additives or pills for treating coprophagy, this hypothesis would also predict that one or more of the commercial products would be beneficial in some instances.

The second hypothesis was that coprophagic dogs may be exhibiting a variant of an innate behavioral predisposition, possibly stemming from wolf ancestors, to help keep the den and rest area free of accumulating feces left in the rest area by an injured or sick wolf where the feces would be a source of intestinal parasite infection if just left alone. The feces could be removed from the den or rest areas by eating right away with minimal risk because the infective forms of intestinal parasites mostly occur in the feces beyond 2 days old. The predictions of this second hypothesis are: coprophagic dogs are as easily housetrained as non-coprophagic dogs (reflecting normal aversion to feces); and coprophagic dogs would tend to consume fresh feces (no more than 2 days old) more than older feces that in nature would contain infective parasite larvae. In contrast to the first hypothesis, another prediction is that this presumably innate predisposition would be very difficult to change by behavior modification approaches or treatment with products specifically marketed for this syndrome.

### SUMMARY

Provided are methods for deterring the ingestion of feces by dogs, e.g., that are frequently showing the tendency to consume their own feces or those of other dogs (by virtue of the wolf-inherited instinct). In one aspect, provided are methods of deterring a canine from ingesting feces. In some embodiments, the method comprises (a) introducing into the interior of a mammalian feces a composition comprising a sufficient amount of lithium chloride (LiCl) to induce nausea in the canine upon or after ingestion of the feces; (b) orally administering or feeding the canine the LiCl-treated or LiCl-impregnated feces, thereby inducing nausea in the canine upon or after ingestion of the feces, wherein the nausea associated with the feces deters the canine from ingesting feces, e.g., at a future time point. In some embodiments, the composition comprising LiCl comprises a concentration in the range of about 0.25 g/ml to about 2.0 g/ml LiCl, e.g., in the range of about 0.5 g/ml to about 1.5 g/ml LiCl, e.g., from about 0.25 g/ml to about 0.5, 0.75, 1.0, 1.25, 1.5, 1.75, or 2.0 g/ml LiCl. In some embodiments, the composition comprising LiCl comprises an aqueous solution or gel. In some embodiments, about 0.5 g to about 3.0 g, e.g., about 0.5 g, 1.0 g, 1.5 g, 2.0 g, 2.5 g, 3.0 g, of LiCl are introduced or impregnated into the interior per about 50 g of feces. In some embodiments, the composition comprising LiCl is not evident to the canine on the surface of the feces. In some embodiments, substantially no LiCl is on the surface of the LiCl-treated or LiCl impregnated feces. In some embodiments, the LiCl-treated feces comprises canine feces, feline feces, equine feces, or mixtures thereof. In some embodiments, the LiCl-treated feces is the canine's own feces. In some embodiments, the LiCl-treated feces is another canine's feces. In some embodiments, the LiCl-treated or LiCl-impregnated feces is fresh, e.g., was expelled within about 96 hours, e.g., within about 72, 48, 36, 24, 12, 6, 3, 2, or 1 hours prior to administration or ingestion by the canine. In some embodiments, the LiCl-treated or LiCl-impregnated feces was frozen within about 96 hours after expulsion, e.g., within about 72, 48, 36, 24, 12, 6, 3, 2, or 1 hours after expulsion. A frozen LiCl-treated or LiCl-impregnated feces is thawed to ambient temperature prior to oral administration or ingestion by the canine. In some embodiments, the feces is a solidly formed feces, e.g., having a texture of a Type 3 or Type 4 Stool as defined on the Bristol Stool Chart. In some embodiments, the methods entail performing steps a) and b) multiple times, e.g., until ingestion of feces by the canine is reduced or inhibited. In some embodiments, the method results in prolonged or permanent aversion by the canine to the ingestion of feces. In some embodiments, the canine is, in whole or in part, from a hound group breed, a terrier group breed, a herding group breed, a sporting group breed, a working group breed, or a toy group breed, as defined by the American Kennel Club (AKC). In some embodiments, the canine is, in whole or in part, from a hound group breed selected from the group consisting of Afghan Hound, American English Coonhound, American Foxhound, Basenji, Basset Hound, Beagle, Black and Tan Coonhound, Bloodhound, Bluetick Coonhound, Borzoi, Cirneco dell'Etna, Dachshund, English Foxhound, Greyhound, Harrier, Ibizan Hound, Irish Wolfhound, Norwegian Elkhound, Otterhound, Petit Basset Griffon Vendeen, Pharaoh Hound, Plott, Portuguese Podengo Pequeno, Redbone Coonhound, Rhodesian Ridgeback, Saluki, Scottish Deerhound, Sloughi, Treeing Walker Coonhound and Whippet. In some embodiments, the canine is, in whole or in part, from a herding group breed selected from the group consisting of Australian Cattle Dog, Australian Shepherd, Bearded Collie, Beauceron, Belgian Malinois, Belgian Sheepdog, Belgian Tervuren, Bergamasco Sheepdog, Berger Picard, Border Collie, Bouvier des Flandres, Briard, Canaan Dog, Cardigan Welsh Corgi, Collie, Entlebucher Mountain Dog, Entlebucher Mountain Dog, Finnish Lapphund, German Shepherd Dog, Icelandic Sheepdog, Miniature American Shepherd, Norwegian Buhund, Old English Sheepdog, Pembroke Welsh Corgi, Polish Lowland Sheepdog, Puli, Pumi, Pyrenean Shepherd, Shetland Sheepdog, Spanish Water Dog and Swedish Vallhund. In some embodiments, the canine is, in whole or in part, from a terrier group breed selected from the group consisting of Airedale Terrier, American Hairless Terrier, American Staffordshire Terrier, Australian Terrier, Bedlington Terrier, Border Terrier, Bull Terrier, Cairn Terrier, Cesky Terrier, Dandie Dinmont Terrier, Glen of Imaal Terrier, Irish Terrier, Kerry Blue Terrier, Lakeland Terrier, Manchester Terrier, Miniature Bull Terrier, Miniature Schnauzer, Norfolk Terrier, Norwich Terrier, Parson Russell Terrier, Rat Terrier, Russell Terrier, Scottish Terrier, Sealyham Terrier, Skye Terrier, Smooth Fox Terrier, Soft Coated Wheaten Terrier, Staffordshire Bull Terrier, Welsh Terrier, West Highland White Terrier and Wire Fox Terrier. In some embodiments, the canine is, in whole or in part, from a sporting group breed selected from the group consisting of American Water Spaniel, Boykin Spaniel, Brittany, Chesapeake Bay Retriever, Clumber Spaniel, Cocker Spaniel, Curly-Coated Retriever, English Cocker Spaniel, English Setter, English Springer Spaniel, Field Spaniel, Flat-Coated Retriever, German Shorthaired Pointer, German Wirehaired Pointer, Golden Retriever, Gordon Setter, Irish Red and White Setter, Irish Setter, Irish Water Spaniel, Labrador Retriever, Lagotto Romagnolo, Nova Scotia Duck Tolling Retriever, Pointer, Spinone Italiano, Sussex Spaniel, Vizsla, Weimaraner, Welsh Springer Spaniel, Wirehaired Pointing Griffon and Wirehaired Vizsla. In some embodiments, the canine is, in whole or in part, from a working group breed selected from the group consisting of Akita, Alaskan Malamute, Anatolian Shepherd Dog, Bernese Mountain Dog, Black Russian Terrier, Boerboel, Boxer, Bullmastiff, Cane Corso, Chinook, Doberman Pinscher, Dogue de Bordeaux, German Pinscher, Giant Schnauzer, Great Dane, Great Pyrenees, Greater Swiss Mountain Dog, Komondor, Kuvasz, Leonberger, Mastiff, Neapolitan Mastiff, Newfoundland, Portuguese Water Dog, Rottweiler, Samoyed, Siberian Husky, Standard Schnauzer, Tibetan Mastiff and St. Bernard. In some embodiments, the canine is, in whole or in part, from a toy group breed selected from the group consisting of Affenpinscher, Brussels Griffon, Cavalier King Charles Spaniel, Chihuahua, Chinese Crested, English Toy Spaniel, Havanese, Italian Greyhound, Japanese Chin, Maltese, Manchester Terrier, Miniature Pinscher, Papillon, Pekingese, Pomeranian, Poodle, Pug, Shih Tzu, Silky Terrier, Toy Fox Terrier and Yorkshire Terrier. In some embodiments, the canine is of a mixed breed or a breed not recognized by the AKC.

In another aspect, provided are compositions, e.g., for use in deterring a canine from ingesting feces. In some embodiments, the compositions comprise mammalian feces comprising in the interior of the feces lithium chloride (LiCl) in an amount sufficient to induce nausea in a canine upon or after ingestion of the feces. In some embodiments, the LiCl-treated or LiCl-impregnated feces comprises canine feces, feline feces, equine feces, or mixtures thereof. In some embodiments, the composition comprises from about 0.5 g to about 3.0 g, e.g., about 0.5 g, 1.0 g, 1.5 g, 2.0 g, 2.5 g, 3.0 g, of LiCl introduced or impregnated into the interior per about 50 g of feces. In some embodiments, LiCl is not evident to the canine on the surface of the feces. In some embodiments, the composition comprises substantially no LiCl on the surface of the feces. In some embodiments, the LiCl-treated or LiCl-impregnated feces was expelled within about 96 hours, e.g., within about 72, 48, 36, 24, 12, 6, 3, 2, or 1 hours. In some embodiments, the LiCl-treated or LiCl-impregnated feces is frozen, and was subject to freezing within about 96 hours after expulsion, e.g., within about 72, 48, 36, 24, 12, 6, 3, 2, or 1 hours, of expulsion. In some embodiments, the fresh-frozen LiCl-treated or LiCl-impregnated feces has been irradiated. In some embodiments, the feces is a solidly formed feces, feces, e.g., having a texture of a Type 3 or Type 4 Stool as defined on the Bristol Stool Chart.

In a related aspect, provided are methods of deterring a canine from ingesting feces. In some embodiments, the methods entail orally administering or feeding the canine the LiCl-treated or LiCl-impregnated feces as described above and herein.

In another aspect, further provided is a syringe. In some embodiments, the syringe comprises a composition comprising LiCl at a concentration in the range of about 0.25 g/ml to about 2.0 g/ml LiCl, e.g., in the range of about 0.5 g/ml to about 1.5 g/ml LiCl, e.g., from about 0.25 g/ml to about 0.5, 0.75, 1.0, 1.25, 1.5, 1.75, or 2.0 g/ml LiCl. In some embodiments, the composition comprising LiCl is an aqueous solution or a gel. In some embodiments, the syringe is a 1 ml, 2 ml, 3 ml or 5 ml syringe. In some embodiments, the syringe has a plastic tip. In some embodiments, the syringe does not comprise a needle. In some embodiments, the syringe is capped with a needle, e.g., a 12 G, 13 G, 14 G, 15 G, 16 G, 17 G or 18 G needle, e.g., having an inner diameter in the range of about 1 mm to about 2 mm. The needle can be blunt or beveled. In some embodiments, the syringe is for use in introducing the composition comprising LiCl into the interior of a feces sample. In some embodiments, the feces is canine feces, feline feces or equine feces. In some embodiments, the feces is a solidly formed feces.

### DETAILED DESCRIPTION

1. Introduction

Provided are compositions, devices and methods for inducing an aversion in canines to eating feces, e.g., canine feces, feline feces, equine feces. In various embodiments, the methods entail impregnating the interior of feces with an amount of lithium chloride (LiCl) sufficient to induce transient or temporary nausea (e.g., nausea that resolves in a matter of hours, analogous to food poisoning) in a canine upon or after ingestion of the LiCl-treated feces.

The survey data from UC Davis provided herein shows that canine stool eating, or coprophagia, occurs on a weekly basis in about 16% of dogs, and while not reflecting any medical abnormality, is very upsetting to dog owners. Some 11 commercial products are marketed for this syndrome, none of which is effective. A great deal of discussion occurs on the internet about this behavior. The compositions, devices and methods described herein are unlike any of the currently commercially available products marketed for use in attempting to curb this stool eating (none of which work).

2. Lithium Chloride (LiCO-Impregnated Stool Compositions

Provided are lithium chloride (LiCl)-impregnated or LiCl-treated stool compositions. Generally, fresh feces, e.g., feces that has been expelled within 4 days, e.g., 3, 2, 1 days, or expelled within 96 hours, e.g., within about 72, 48, 36, 24, 12, 6, 3, 2, or 1 hours is impregnated with an amount of a lithium chloride composition sufficient to induce transient nausea in a canine upon or after ingestion of the LiCl-impregnated or LiCl-treated feces. The feces can be canine feces (e.g., the canine's own feces or another canine's feces), feline species (e.g., in a household litter box), equine feces, or mixtures thereof. When preparing stool compositions from canine feces, generally the feces has been expelled within 2 days, e.g., within 1.5 or 1 days, or expelled within 48 hours, e.g., within about 36, 24, 12, 6, 3, 2, or 1 hours.

In some embodiments, the composition comprises from about 0.5 g to about 3.0 g, e.g., about 0.5 g, 1.0 g, 1.5 g, 2.0 g, 2.5 g, or 3.0 g, of LiCl introduced or impregnated into the interior per about 50 g of feces. Stool samples that are smaller than about 50 g (e.g., from a small canine or from a feline) can be impregnated with smaller amounts of the LiCl composition. Stool samples that are larger than about 50 g (e.g., from a large dog), can be reduced to a mass of about 50 g. The LiCl composition can be introduced or impregnated into the interior of the feces sample at one or more locations. In some embodiments, the LiCl composition is injected or impregnated below the surface at several different locations in a single stool sample, e.g., spaced 1, 2 or 3 cm apart. In order that the canine does not detect that a stool has been treated with LiCl, the LiCl composition is injected or impregnated below the surface of the stool, such that that there is substantially no LiCl composition on the surface of the stool. In various embodiments, the LiCl composition injected into or impregnated into the stool is an aqueous solution or a gel, e.g., having a viscosity of about 500 centipoise (cps) or less

In some embodiments, the LiCl-treated or LiCl-impregnated feces is frozen, and was subject to freezing within about 96 hours after expulsion, e.g., within about 72, 48, 36, 24, 12, 6, 3, 2, or 1 hours, of expulsion. In some embodiments, the fresh-frozen LiCl-treated or LiCl-impregnated feces has been irradiated.

Generally, stool samples suitable for LiCl-treatment or LiCl-impregnation are solidly formed stools. For example, in some embodiments, stools suitable for LiCl-treatment or LiCl-impregnation have a texture of a Type 3 or Type 4, as defined by the Bristol Stool Form Scale (BSFS), originally described by O'Donnell and Heaton, Gut (1988) 29: A1455 and reviewed in Perez and Martinez, Rev Esp Enferm Dig. (2009) May;101(5):305-11. In certain embodiments, the LiCl-treated stool compositions can be prepared from stools having the texture of Type 2, Type 3, Type 4 or Type 5 stool, as defined by the BSFS. According to the Bristol Stool Form Scale, seven types of stool are:

Type 1: Separate hard lumps, like nuts (hard to pass); also known as goat feces

Type 2: Sausage-shaped, but lumpy

Type 3: Like a sausage but with cracks on its surface

Type 4: Like a sausage or snake, smooth and soft

Type 5: Soft blobs with clear cut edges (passed easily)

Type 6: Fluffy pieces with ragged edges, a mushy stool

Type 7: Watery, no solid pieces, entirely liquid

3. Methods of Deterring Canine Coprophagy

Provided are methods of deterring a canine from ingesting feces. In some embodiments, the method comprises (a) introducing into the interior of a mammalian feces a composition comprising a sufficient amount of lithium chloride (LiCl) to induce temporary nausea in the canine upon or after ingestion of the feces; (b) orally administering or feeding the canine the LiCl-treated or LiCl-impregnated feces, thereby inducing nausea in the canine upon or after ingestion of the feces, wherein the nausea associated with the feces deters the canine from ingesting feces. In some embodiments, the methods entail orally administering or feeding to a canine a LiCl-impregnated stool composition, as described above and herein. Without being bound to theory, by inducing nausea in the canine through administration of LiCl-treated feces, the canine will develop an association of nausea with the ingestion of feces and therefore a long-term or permanent aversion to ingestion of feces.

a. Impregnating Stool Sample with LiCl Composition

With respect to the step of preparing LiCl-impregnated or LiCl-treated stool, a composition comprising a sufficient amount of LiCl to induce transient or temporary nausea in the canine upon or after ingestion of the feces is introduced into the interior (i.e., below the surface) of a stool sample. In various embodiments, the composition comprising LiCl comprises a concentration in the range of about 0.25 g/ml to about 2.0 g/ml LiCl, e.g., in the range of about 0.5 g/ml to about 1.5 g/ml LiCl, e.g., from about 0.25 g/ml to about 0.5, 0.75, 1.0, 1.25, 1.5, 1.75, or 2.0 g/ml LiCl. The LiCl-impregnated or LiCl-treated feces compositions can be prepared as described above and herein.

b. Orally Administering or Feeding LiCl-Impregnated Stool to Canine

The LiCl-impregnated or LiCl-treated feces compositions are then orally administered or fed to the canine. As used herein, oral administration does not refer to force feeding. The canine is allowed to self-administer by voluntary ingestion the LiCl-impregnated or LiCl-treated feces. In embodiments using fresh frozen LiCl-treated or LiCl-impregnated feces, the LiCl-treated or LiCl-impregnated feces is thawed to ambient temperature prior to oral administration or ingestion by the canine. During the time period for treatment, the canine being treated is not exposed to or allowed to ingest feces that have not been LiCl-treated or LiCl-impregnated. Ingestion of the LiCl-treated or LiCl-impregnated feces induces temporary or transient nausea and in some instances vomiting by the canine.

In some embodiments, the methods entail performing the steps of preparing LiCl-treated or LiCl-impregnated feces and/or oral administration or feeding of LiCl-treated or LiCl-impregnated feces (e.g., when using previously prepared LiCl-treated or LiCl-impregnated feces feces) multiple times as needed, e.g., until ingestion of feces by the canine is reduced or inhibited. In some embodiments, the method results in prolonged or permanent aversion by the canine to the ingestion of feces. If the canine reverts to ingestion of feces, the steps can be repeated as necessary. The method is discontinued if the canine exhibits symptoms of extended, serious or life-threatening illness.

The methods find use in treating any canine inclined to coprophagy, including dogs of mixed breed or a recognized breed, although certain dog breeds have a greater propensity than others to ingest feces. In some embodiments, the canine is, in whole or in part, from a hound group breed, a terrier group breed, a herding group breed, a sporting group breed, a working group breed, or a toy group breed, as defined by the American Kennel Club (AKC). Dog breed groups are listed at http://www.akc.org/dog-breeds/groups/. In some embodiments, the canine is of a mixed breed or a breed not recognized by the AKC.

Illustrative canines from hound group breeds include without limitation Afghan Hound, American English Coonhound, American Foxhound, Basenji, Basset Hound, Beagle, Black and Tan Coonhound, Bloodhound, Bluetick Coonhound, Borzoi, Cirneco dell'Etna, Dachshund, English Foxhound, Greyhound, Harrier, Ibizan Hound, Irish Wolfhound, Norwegian Elkhound, Otterhound, Petit Basset Griffon Vendeen, Pharaoh Hound, Plott, Portuguese Podengo Pequeno, Redbone Coonhound, Rhodesian Ridgeback, Saluki, Scottish Deerhound, Sloughi, Treeing Walker Coonhound and Whippet.

Illustrative canines from herding group breeds include without limitation Australian Cattle Dog, Australian Shepherd, Bearded Collie, Beauceron, Belgian Malinois, Belgian Sheepdog, Belgian Tervuren, Bergamasco Sheepdog, Berger Picard, Border Collie, Bouvier des Flandres, Briard, Canaan Dog, Cardigan Welsh Corgi, Collie, Entlebucher Mountain Dog, Entlebucher Mountain Dog, Finnish Lapphund, German Shepherd Dog, Icelandic Sheepdog, Miniature American Shepherd, Norwegian Buhund, Old English Sheepdog, Pembroke Welsh Corgi, Polish Lowland Sheepdog, Puli, Pumi, Pyrenean Shepherd, Shetland Sheepdog, Spanish Water Dog and Swedish Vallhund.

Illustrative canines from terrier group breeds include without limitation Airedale Terrier, American Hairless Terrier, American Staffordshire Terrier, Australian Terrier, Bedlington Terrier, Border Terrier, Bull Terrier, Cairn Terrier, Cesky Terrier, Dandie Dinmont Terrier, Glen of Imaal Terrier, Irish Terrier, Kerry Blue Terrier, Lakeland Terrier, Manchester Terrier, Miniature Bull Terrier, Miniature Schnauzer, Norfolk Terrier, Norwich Terrier, Parson Russell Terrier, Rat Terrier, Russell Terrier, Scottish Terrier, Sealyham Terrier, Skye Terrier, Smooth Fox Terrier, Soft Coated Wheaten Terrier, Staffordshire Bull Terrier, Welsh Terrier, West Highland White Terrier and Wire Fox Terrier.

Illustrative canines from sporting group breeds include without limitation American Water Spaniel, Boykin Spaniel, Brittany, Chesapeake Bay Retriever, Clumber Spaniel, Cocker Spaniel, Curly-Coated Retriever, English Cocker Spaniel, English Setter, English Springer Spaniel, Field Spaniel, Flat-Coated Retriever, German Shorthaired Pointer, German Wirehaired Pointer, Golden Retriever, Gordon Setter, Irish Red and White Setter, Irish Setter, Irish Water Spaniel, Labrador Retriever, Lagotto Romagnolo, Nova Scotia Duck Tolling Retriever, Pointer, Spinone Italiano, Sussex Spaniel, Vizsla, Weimaraner, Welsh Springer Spaniel, Wirehaired Pointing Griffon and Wirehaired Vizsla.

Illustrative canines from working group breeds include without limitation Akita, Alaskan Malamute, Anatolian Shepherd Dog, Bernese Mountain Dog, Black Russian Terrier, Boerboel, Boxer, Bullmastiff, Cane Corso, Chinook, Doberman Pinscher, Dogue de Bordeaux, German Pinscher, Giant Schnauzer, Great Dane, Great Pyrenees, Greater Swiss Mountain Dog, Komondor, Kuvasz, Leonberger, Mastiff, Neapolitan Mastiff, Newfoundland, Portuguese Water Dog, Rottweiler, Samoyed, Siberian Husky, Standard Schnauzer, Tibetan Mastiff and St. Bernard.

Illustrative canines from toy group breeds include without limitation consisting of Affenpinscher, Brussels Griffon, Cavalier King Charles Spaniel, Chihuahua, Chinese Crested, English Toy Spaniel, Havanese, Italian Greyhound, Japanese Chin, Maltese, Manchester Terrier, Miniature Pinscher, Papillon, Pekingese, Pomeranian, Poodle, Pug, Shih Tzu, Silky Terrier, Toy Fox Terrier and Yorkshire Terrier.

4. Syringes Loaded with Lithium Chloride (LiCl) Solution or Gel

In another aspect, further provided is a syringe, e.g., for use in introducing the composition comprising LiCl into the interior of a feces sample. In some embodiments, the syringe comprises a composition comprising LiCl at a concentration in the range of about 0.25 g/ml to about 2.0 g/ml LiCl, e.g., in the range of about 0.5 g/ml to about 1.5 g/ml LiCl, e.g., from about 0.25 g/ml to about 0.5, 0.75, 1.0, 1.25, 1.5, 1.75, or 2.0 g/ml LiCl. In some embodiments, the composition comprising LiCl is an aqueous solution or a gel. When prepared as a gel, the LiCl composition has a viscosity that is sufficiently low such that the gel can be readily pushed out of the syringe, with or without a needle, into the interior of a stool sample. In various embodiments, the LiCl gel composition has a viscosity of about 500 centipoise (cps) or less.

In some embodiments, the syringe is a 1 ml, 2 ml, 3 ml or 5 ml syringe. In some embodiments, the syringe has a plastic tip. In some embodiments, the syringe does not comprise a needle. In some embodiments, the syringe is capped with a needle, e.g., a 12 G, 13 G, 14 G, 15 G, 16 G, 17 G or 18 G needle, e.g., having an inner diameter in the range of about 1 mm to about 2 mm The needle can be blunt or beveled.

5. Kits

Further provided are kits. In some embodiments, the kits comprise one or more syringes, as described above and herein. In some embodiments, the kits comprise multiple syringes that can be preloaded with an LiCl composition, as described above and herein. The kit may further comprise one or more containers comprising the LiCl composition prepared in 1× concentration (ready for use), or as concentrate (e.g., 2×, 3×, 4×, 5×, or more). In some embodiments, the kits comprise one syringe and a container comprising the LiCl composition prepared in 1× concentration (ready for use), or as concentrate (e.g., 2×, 3×, 4×, 5×, or more). The kits will further comprise instructions for preparing LiCl-treated or LiCl-impregnated feces and for oral administration or feeding to the canine, as described above and herein.

In some embodiments, the kits comprise one or more fresh, frozen LiCl-treated or LiCl-impregnated stool samples, as described above and herein. The fresh, frozen LiCl-treated or LiCl-impregnated stool samples can be canine, feline or equine feces or mixtures thereof. Such fresh, frozen LiCl-treated or LiCl-impregnated stool samples may be irradiated. The kits will further comprise instructions for thawing the fresh, frozen LiCl-treated or LiCl-impregnated feces and for oral administration or feeding to the canine, as described above and herein.

### EXAMPLES

The following examples are offered to illustrate, but not to limit the claimed invention.

### Example 1

### The Paradox of Canine Conspecific Coprophagy: Commercially Available Deterrents Do Not Work

### Abstract

Canine conspecific coprophagy, the tendency or predisposition of some dogs to eat their own feces or those of other dogs, seems paradoxical because dogs typically show an aversion to conspecific feces. In an attempt to resolve this paradox, we set out to determine the factors associated with the occurrence of this behavior and to evaluate the efficacy of 11 products marketed for treating coprophagy as well as behavior modification procedures.

Because a large sample of dogs was needed to address these issues, two web-based surveys were utilized. One, intended to compare coprophagic dogs and non-coprophagic dogs, yielded 1,552 returns. The other, yielding 1,475 usable returns, specifically recruited owners of coprophagic dogs to gather information about the characteristics of coprophagy and treatment success.

The findings revealed that 16 percent of dogs sampled engaged in frequent conspecific coprophagy, defined as having been seen eating stools at least 6 times. No evidence was found relating the coprophagy to diet or the dog's age. Coprophagic dogs were as easily house trained as non-coprophagic dogs, suggesting a normal aversion to feces. Coprophagic dogs were more likely to be reported as greedy eaters than non-coprophagic dogs. The reported success rate of the commercial products and behavior modification approaches was close to zero, indicating that the behavior is not readily changed.

The coprophagy was overwhelmingly directed at fresh stools, defined as being no more than 2 days old. An hypothesis is offered that coprophagy reflects a tendency inherited from the ancestral wolf to keep the den area free of fecal-borne intestinal parasites that might be deposited in the den rest area and would typically have parasite ova that are not initially infective, but could develop infective larvae after 2 days. An evolved parasite defense strategy to consume fresh feces in the rest area would be adaptive.

### Methods

Data collection. The types of information sought in this study required a large database, far beyond what one could obtain by interviewing dog owners. From past studies at this center, and knowing that statistical analyses for the information sought would require responses from at least 1,000 dog owners, two web-based surveys were designed, carefully planned and pilot-tested. Similar web-based surveys have been used in a variety of data-based behavioral and medical publications (11-15), and have been shown to provide data of a quality and validity comparable to traditional paper and pencil survey methods (16-18). The self-administered surveys were intended to take 10-15 minutes of the respondents' time.

Launched in 2010-11, the surveys were completed anonymously and voluntarily, with no personal identifiers, by interested dog owners recruited on dog listservs. As with other published and anonymous web-based surveys from this center, there was no follow-up contact with those responding to the survey, and only the anonymous data responses to the survey were viewed. Therefore, no human subject committee approval was needed for such data use.

The two surveys were launched 6 months apart (SurveyMonkey®). One was intended to estimate the prevalence of coprophagy and compare coprophagic dogs and non-coprophagic dogs, and was labeled, “Dog Behavior: The Rest of the Story.” The title and introductory information did not mention stool eating, and the specific stool-eating questions were imbedded in a series of questions about the dog's diet, eating behavior and behaviors not relevant to coprophagy. The criterion for being coprophagic in this survey was that the dog had been seen eating stools at least 6 times. Table 1 provides a summary of question categories in this survey.

TABLE 1
Survey 1. Dog Behavior, the Rest of the Story.
Categories of Questions

Demographic data, such as number of dogs in household, sex, age, breed
of dog
Yard space available to dogs
Ease of housetraining
Type of food given
Type of eater: finicky; greedy; normal
Dog's level of affection
Problem behaviors the dog has from list of 10
Howling at sirens
Eating non-nutritional material other than grass or stools of dogs
Frequency of grass or plant eating
For stool eaters: frequency of eating stools: daily; weekly; monthly; yearly
For stool eaters: dog mostly eats only own stools; only stools of other
dogs; both
For stool eaters: age of stools mostly eaten: 1-2 days; 2-4 days; >4 days

The second survey was labeled “Why Dogs Eat Their Stools,” and owners of coprophagic dogs were intentionally recruited so as to obtain detailed information on dogs that were well-known by their owners to be frequently coprophagic. Questions sought information on the age of stools that dogs consumed, the frequency of the behavior and success in resolving the behavior using products marketed for the problem and success with behavior modification approaches. There was some overlap in questions between the two surveys, providing a cross-check in reliability. Table 2 provides a summary of question categories in this survey. The criterion for keeping responses in the database was that the dog was seen eating stools more than 10 times and at least once a month to increase the likelihood that the responses regarding the details of stool eating were accurate.

TABLE 2
Survey 2. Why Dogs Eat Stools.
Categories of Questions

Demographic data, such as number of dogs in household, sex, age, breed
of dog
Ease of housetraining
Type of food given
Type of eater: finicky; greedy; normal
Dog's level of affection
Problem behaviors the dog has from list of 10
Frequency of grass or plant eating
Age that stool eating first noticed
Total times observed eating stools
Whether dog mostly eats only own stools; only stools of other dogs; both
Age of stools mostly eaten: 1-2 days; 2-4 days; >4 days
Frequency of eating stools: daily; weekly; monthly; yearly
Ways that you know a stool was eaten
Behavior modification treatments tried from a list of 7 and success of
treatment
Commercial treatments tried from a list of 11 and success of treatment

Statistical Analyses. Chi-square tests and Fisher's Exact tests for non-parametric comparisons were used for pairwise comparisons between dogs specified as coprophagic, having been seen eating stools at least 6 times, and non-coprophagic dogs, designated as never having been seen eating stools. The level of significance was set at p<0.05, two-tailed, and the Chi-square value is given. In all cases, the Chi-squared and Fisher tests produced qualitatively identical results, so just the Chi-square values are given. The logistic regression analysis was a stepwise logistic regression including only the variables that had a significance of p≤0.01. This value was chosen to reduce the likelihood of a false positive of a variable. All analyses were run using SAS, version 9.4 (SAS Institute, Cary, N.C.).

### Results

Prevalence of conspecific coprophagia in dogs and demographics. The data for this section were from the survey, “Dog Behavior: The Rest of the Story,” where a total of 1,552 useable responses were returned before the survey was closed and the data were gathered for analyses. The returns from the surveys came overwhelmingly from the US (89.8 percent) and Canada (5.1 percent). For multi-dog households, the respondents were told to choose the dog they knew best, or had known the longest, for answering questions; this was referred to as the specified dog. The specified dog could not be a mother with puppies, where some stool eating might be expected. Of the 1,441 respondents answering the questions about conspecific stool eating, 76.9 percent (1,108) reported never having seen their dog eating stools (referred to as non-stool eaters or non-coprophagic), while 16.0 percent (230) reported having seen their dogs eating stools ≥6 times (referred to as frequent stool eaters or coprophagic). Those reporting having seen their dogs eating stools 1-5 times (classified as neither coprophagic nor non-coprophagic) were 7.1 percent (103). Accordingly, about 23 percent of the dogs sampled reportedly were seen eating stools at least 1 time. Depending upon how one categorizes a stool eater, i.e., seen ≥6 times, or ≥1 time, the prevalence of stool eating among dogs represented by this survey ranged from 16 percent to 23 percent. Of the respondents with frequent stool eaters, 79.6 percent reported seeing their dogs eating stools greater than 10 times.

To make the contrast between coprophagic and non-coprophagic dogs clear in the results presented below, unless otherwise noted, only dogs that were never seen eating stools were compared with dogs seen eating stools at least 6 times. We prepared an extensive table showing responses to each of the questions with all responses, and where dogs referred to as coprophagic are compared with non-coprophagic dogs. In this survey 82 percent of coprophagic dogs were described as consuming stools that were no more than 2 days old.

The occurrence of coprophagy was distributed among all four gender-neuter groups and this measure did not distinguish between frequent stool eaters and non-stool eaters. The distribution for neutered males, spayed females, intact males and intact females was 45.2, 41.7, 6.1, and 7.0 percent respectively for stool eaters and 41.3, 40.7, 10.2 and 7.8 percent for non-stool eaters. Coprophagy does not seem to be a reflection of juvenile behavior. Of coprophagic dogs 1.7 percent were less than 1 year of age and 75.1 percent were over 4 years of age, compared with non-coprophagic dogs of which 3.2 percent were less than 1 year of age and 69.7 percent were over 4 years of age. Coprophagy does not seem to be related to age of separation from the dam. Of coprophagic dogs 59.1 percent were reported as being left with the dam for at least 7 weeks compared with 49.7 percent of non-coprophagic dogs. Diet appears not to be related to coprophagy in that for 82.3 percent of frequent stool eaters, and 78.3 percent of non-eaters, kibble was the main food.

An indication that coprophagy does not reflect a weak aversion to feces is that 78 percent of dogs that were frequent stool eaters had been easily housetrained and remained well house trained, and a similar 82 percent of non-stool eaters fell into this category of house training.

Coprophagy does not seem to be associated with the occurrence of compulsive-like behaviors. Compulsive-like behaviors were noted in 3.5 percent of frequent stool eaters and 2.9 percent of non-eaters. The list of problem behaviors that could be noted, in addition to compulsive-like behaviors by the responders, included separation anxiety, various types of aggressive behavior, destructive behavior, and excessive barking, none of which were related to coprophagy.

Several variables were statistically associated with coprophagy, and Table 3 presents a stepwise logistic regression analysis of factors significantly related to coprophagy. The variable most highly associated with coprophagy was the reported eating style, with 51.1 percent of coprophagic dogs referred to as greedy eaters compared with just 28.2 percent of non-coprophagic dogs.

TABLE 3
Stepwise Logistic Regression Analyses of Factors
Related to Coprophagy.*

| Factor | Parameter | Chi sq | P-value |
|---|---|---|---|
| Greedy eating | 0.86 | 27.90 | <0.0001 |
| Breed Group | NA | 20.79 | 0.0077 |
| Multiple dogs in | 0.62 | 11.07 | 0.0005 |
| household | | | |
| Eating dirt | 1.7 | 14.72 | 0.0001 |
| Eating cat stools | 0.51 | 10.84 | 0.001 |

*See text for more details. The parameter measure for breed group evaluated 9 different breed groups, each with a different value, so this is indicated as NA.

Breed identification was considered in two respects. One was with regard to breed group. In Table 3 of the logistic regression analysis, it can be seen that terriers and hounds are most likely to be coprophagic. With regard to specific breeds, the database, even with 1,552 responses, could only provide limited information on individual breeds occurring frequently enough for comparisons. The specific breeds examined in this regard were those for which there were at least 15 dogs that met the criterion of being either coprophagic or non-coprophagic. Shetland Sheepdogs (N=27), with 41 percent being coprophagic, were overrepresented in comparison to 17 percent of other breeds (p=0.003). On the other hand, with pooling the 3 varieties of Poodles (Standard, Miniature, and Toy) (N=29), none of the dogs was coprophagic (p=0.006). Given that about 90 percent of the responses came from the US, and the presumed differences in dog breeds between the US and other countries, the information about breed groups or specific breeds is offered as relevant mostly to the US.

The number of dogs in the household was important with dogs living in households with two or more dogs most likely to be coprophagic. While eating non-nutritional substances was asked about in the survey, there was no question about the opportunity of the dog to eat the various non-nutritional substances, such as horse or cattle stools. However, because dirt and cat stools would be frequently around, these were left in the logistic regression analysis. Having been reported as eating dirt and eating cat stools were positively associated with coprophagy.

Specific characteristics of coprophagia. The data for this section were from the survey entitled, “Why Dogs Eat Their Stools.” There were 2,561 returns before the survey was closed. Inclusion criterion were then applied, one being that the dog had to have been seen eating stools greater than 10 times. This criterion was posed in a question giving several options: 1-5 times, 6-10 times, and greater than 10 times. A second criterion applied was that stool eating had to have been observed at least on a weekly basis. This criterion was posed in a question giving several options: daily, weekly, monthly, yearly and less than once a year. These rather demanding criteria were considered necessary to focus on dogs that were reliably coprophagic and where the behavior was observed frequently. The survey with these criteria yielded 1,475 returns, of which 62 percent ate stools daily and 38 percent weekly.

Coprophagic dogs from this survey were 30 percent neutered males, 42 percent spayed females, 9 percent intact males and 19 percent intact females. In this survey, 74 percent of the dogs had been housetrained easily, similar to the 78 percent of frequent stool eaters being easily housetrained in the first survey. With regard to eating style, in this survey, 52 percent were referred to as greedy eaters, compared to an almost identical 51 percent of frequent stool eaters in Survey 1 being referred to as greedy eaters.

The coprophagic dogs in this survey primarily consumed stools that were no more than 2 days old—referred to as fresh stools—with 85 percent identified as eating fresh stools. This corresponds to Survey 1 with 82 percent of frequent stool eaters having been seen eating stools no older than 2 days. The two surveys, taken together, confirm that coprophagic dogs overwhelmingly consume fresh stools.

An important finding in this survey is that the coprophagy appears not to have been altered by either behavior modification and/or management techniques attempted by caregivers. In descending order of frequency of use, the various procedures were: chase away from stools (n=1,048); reward the successful command of “leave it alone” (n=424); lace stools with pepper (n=295); and punish by electronic or sound-emitting collar (n=56). The reported success rate was 1-2 percent except for “leave it alone” which was slightly higher at 4 percent.

The responses regarding the success of the 11 food additives or tablets specifically advertised for treatment of coprophagy are given in Table 4. While we have no information about the degree to which respondents followed instructions given with the products, coprophagy appeared not to be meaningfully altered by any of the products. The number of responders using these products ranged per product from 6 to 352. The reported rate of success ranged from 0-2 percent.

TABLE 4
Food Additives and Pills Marketed for Coprophagia.†

| | Responses | Percent |
|---|---|---|
| Name of Product | for Product | Reporting Success |
| For-Bid ® | 352 | 1 |
| Deter ® | 238 | 1 |
| Dis-Taste ® | 154 | 1 |
| Coproban ® | 58 | 2 |
| S.E.P ® | 58 | 0 |
| Stop Stool Eat ® | 27 | 0 |
| Stop Tablets ® | 26 | 0 |
| Potty Mouth ® | 24 | 0 |
| NaturVet Deter ® | 20 | 0 |
| Nasty Habit ® | 13 | 0 |
| 21st Century ® | 6 | 0 |

†Responders were given a list of products and asked to say if the stool eating in their specified dog was resolved. Shown are the 11 products found, as of the writing of the paper. Some of these are dispensed through a veterinarian and some sold over the counter. The survey did not explore the degree to which the respondent closely followed directions on the label.

### Discussion

As indicated by the internet attention given to this behavior, canine conspecific coprophagy is, undeniably, an important concern for owners of companion dogs. One purpose of this study was to estimate the prevalence of conspecific coprophagy in the general population of dogs, as well as determine what environmental, biological, and management factors might differentiate coprophagic dogs from those that are not coprophagic. Another purpose was to explore the frequency of stool eating for those that are coprophagic, the age of stools eaten, and the success of dog owners in using various behavior modification techniques to eliminate this behavior as well as their success with any of the 11 products marketed specifically for treating canine coprophagy. Finally, there was a goal to offer a hypothesis for the occurrence of canine conspecific coprophagy in a broad population of dogs.

One finding from the first web-based survey with 1552 usable responses was that 16 percent of dogs in general are coprophagic, defined as having been observed eating stools at least 6 times. The occurrence of dogs seen eating dog stools at least once was 23 percent. Thus, depending on how one defines canine coprophagy, the occurrence is between 16 and 23 percent. In this paper a coprophagic dog is defined as one having been seen eating dog stools at least 6 times, and a non-coprophagic dog as one never having been seen eating dog stools.

In contrasting coprophagic with non-coprophagic dogs, it was found that there was no difference with regard to distribution among sex or neuter categories, age, diet, ease of house training, or association with a compulsive behavior. Coprophagy could not be ascribed to a lack of normal mothering, because where information was available, coprophagic dogs were as likely to have been left with the dam for over 7 weeks as non-coprophagic dogs.

Several factors did, however, distinguish between coprophagic and non-coprophagic dogs in a significant manner with a significance value of p<0.01 (chosen to reduce the likelihood of false positives). Coprophagic dogs were much more likely to be described as greedy eaters. Coprophagic dogs were more likely to be found in multi-dog households presumably where there would be a greater concentration of stools. Eating dirt and cat stools were positively associated with coprophagy. Breed group (American Kennel Club designation) was associated with coprophagy, with terriers and hounds most positively associated with coprophagy. Of individual breeds for which sufficient numbers were reported, Shetland Sheepdogs were overrepresented and Poodles (all varieties) underrepresented.

With regard to specific information on coprophagy from the second survey with 1,475 responses meeting the criteria where the dogs were seen eating stools over 10 times and at least once a week, 85 percent were reported to eat stools no more than 2 days old (fresh stools). Similar results were found in the first survey, confirming that coprophagic dogs overwhelmingly consume fresh stools.

The success in eliminating the coprophagia with the various behavioral procedures ranged from only 1 to 4 percent. The survey did not request information on whether the behavioral techniques were directed from a canine behavior specialist or were just tried on the respondents' own initiative. The reported success rate for food additives or tablets marketed for coprophagy ranged from 0-2 percent. The survey did not get into the extent to which the respondent did or did not follow instructions which may have come with the product.

Two contrasting hypotheses were formulated for explaining coprophagy. One is that coprophagic dogs are exhibiting an abnormal behavior stemming from one or more contributing causes. The second is that coprophagy is an expression of an adaptive behavior inherited from ancestral wolves. None of the findings reviewed above supported the first hypothesis. Coprophagic dogs seemed to be as easily house trained as non-coprophagic dogs, which we assume is an indication of aversion to feces. Therefore, we do not ascribe coprophagy to an abnormal lack of aversion to feces.

The perspective of the second hypothesis refers to an adaptive behavioral defense against parasites of wolves living in nature where feces of injured or sick pack members might be deposited in the rest areas near the den. If wolves were to remove the feces from rest areas where infective larvae from intestinal parasites would become more numerous over time, consumption is the only method available. For the most frequently reported intestinal parasites in wolf feces, larvae from ova expelled in feces, do not develop into infective forms, that can directly transmit parasites, for at least 2 days.

A list of the reported intestinal parasites found in scats of wolves, with development times in feces (19), is shown in Table 5. Table 5 lists a classification of different genera of intestinal parasites found in dogs and in scats of wild wolves [4-8]. In wild canids, several species are generally mentioned. The various parasite species of each type have the same basic life cycle. Note that the various parasite types require either an intermediate host before being infective, or require at least 2 days of development in the feces, before being infective to the canid that is the definitive host. Life cycle information summarized from various resources (19).

### Table 5. Intestinal Parasites Found in Scats of Wild Wolves and Other Canids.

Helminths (parasitic worms)

Cestodes (tapeworms)
Taenia spp., Echinococcus spp. Require ingestion by an intermediate host before infecting definitive host

Trematodes (flatworms or flukes)
Alaria spp. Require two intermediate hosts in water. Common in wolves, not dogs

ematodes (roundworms)
Ancylostomidae and Uncinaria spp. Hook worms. Larvae develop into infective forms in the ova 2-9 days after being shed in the feces. Infective larvae then develop in the environment (soil) and can then penetrate the skin of hosts.
Trichuris spp. Whipworms. Infective larvae develop in ova of shed feces after 10-25 days
Toxocara spp. Ascarids. Infective larvae develop in ova in shed feces in 2-4 weeks
Strongyloides spp. Pinworms or threadworms. Infective larvae hatch from ova in feces in 2-3 days, and typically infect through the skin. Infections are usually mild.
Coccidea (single cell parasites)
   Isospora spp. Oocysts develop into infective sporulated oocysts in feces in 3-5 days

For the parasites in the feces, the ova passed in the shed feces develop into infective larvae after a few days, depending upon parasite species and ambient temperatures. Leaving the feces alone would allow the ova to hatch into infective larvae that could be picked up on the hair of wolves and groomed off, thus transmitting the parasites. If the feces are consumed while fresh, however, within about 2 days, the larvae will not yet have developed into infective forms and the risk is presumably much less.

There are other, less frequently occurring parasites found in canid feces, as well as pathogenic bacteria from sick individuals, that can be immediately infective, so the consumption of fresh feces can be considered “the lesser of two evils”—leaving them alone or immediately consuming them.

This hypothesis that some domestic dogs could be displaying the wolf-like coprophagy behavior is a parallel to other wolf-like behavioral predispositions seen in dogs. In fact the finding that being a “greedy eater” is the strongest differentiating variable associated being a coprophagic dog, would seem to support this wolf origin of coprophagy because one would expect greedy eating to be a common wolf characteristic.

We are aware of no study on wolves (or other canids) in nature that involves data-based observations of stool eating in the rest areas. Indeed, according to the perspective presented here, one would expect this behavior not only to be infrequent, but to be carried out swiftly so that an observer could easily miss seeing it. However, a comment by noted wolf authority L. David Mech that “wolves do commonly practice coprophagy, at least in captivity” (20, p. 101), offers support for this perspective, which was further reinforced by a personal communication with Mech.

In the current environment, intestinal parasites of domestic dogs are commonly prevented and/or treated by anthelmintics, which could result in relaxed natural selection for behaviors that would be related to avoidance of intestinal parasites. Consequently, one would expect some dogs to be vigilant in consuming stools, others to have completely lost this behavior and others to be stool eaters on a sporadic basis. Additional variability in coprophagy would have come about in the selective breeding of dogs over centuries where one could expect differences in selection against this behavior. Consistent with this perspective, we found an apparent under-representation of coprophagy in Poodles and over-representation in Shetland Sheepdogs. We are not aware of any publicized comments in breed development literature that may have played a role in the selection against coprophagy, but given the repulsive nature of the behavior to humans, one would expect some breeders to avoid breeding dogs that are frequently coprophagic.

While the coprophagic syndrome seems to be medically harmless, it is very disturbing for many dog owners. One publication discussing this syndrome notes that some people find it so disgusting that the bond with their dog is irreparably damaged to the point where euthanasia is considered (21).

There are several caveats that need to be mentioned in this study. One is with regard to the finding of no successful results in treating coprophagy with any of the commercial products. To our knowledge, there have been no clinical trials with designated procedures for recruiting subjects to be treated and assuring that treatment guidelines were followed. Thus, there could be treatments that would be effective if instructions were followed. Secondly, the rule-out of a compulsive disorder could be studied by careful observations on a sample of frequently coprophagic dogs and treatment with a psychotropic medication considered effective for some compulsive disorders. Thirdly, the explanation referring to a hypothetical parasite defense of wolf ancestors has no substantiating field observations and should be considered tentative.

### REFERENCES

1. Hart B L, Hart L A, Bain M J (2006) Canine and Feline Behavior Therapy, second ed. Blackwell, Ames, Iowa.
2. Hart B L (1990) Behavioral adaptations to pathogen and parasites: Five strategies. Neurosci Biobehav Rev 14: 273-294.
3. Hart B L (2012) Behavioral defenses in animals against pathogens and parasites: parallels with the pillars of medicine in humans Phil Tran Roy Soc B 366: 3406-3417.
4. Bynum D, van Ballenberghe V, Schlotthauer J C, Erickson A W (1977) Parasites of wolves, Canis lupus L., in northeastern Minnesota, as indicated by analysis of fecal samples. Can J Zool 55: 376-380.
5. Custer J W, Pencet D B (1981) Ecological analyses of helminth populations of wild canids from the gulf coastal prairies of Texas and Louisiana. J Parasitol 67: 289-300.
6. Kloch A, Bajer A (2005) Intestinal macro- and microparasites of wolves (Canis lupis L.) from north-eastern Poland recovered by coprological study. Ann Agric Environ Med 12: 237-245.
7. Marquard-Peterson U (1997) Endoparasites of Arctic Wolves in Greenland. Artic 50: 349-354.
8. Stancampo G V, Francisci F (1993) Intestinal helminth parasite community in wolves (Canis lupus) in Italy. Parasitologia 35: 59-65.
9. Boze B (2008) A comparison of common treatments for coprophagy in Canis familiaris. J Appl Comp Anim Behav 2: 22-28.
10. Wells D L (2003) Comparison of two treatments for preventing dogs eating their own faeces. Vet Rec 153: 51-53.
11. Gobar G M, Kass P H (2002) Worldwide web-based survey of vaccination practices, postvaccinational reactions, and vaccine site-associated sarcomas in cats. J Am Vet Med Assoc 220: 1425-1576.
12. Janson C, Wist M (2004) An internet survey of asthma treatment. J Asthma 41: 44-55.
13. McCobb E C, Brown E A, Damiani K, Dodman N H (2001) Thunderstorm phobia in dogs: an internet survey of 69 cases. J Am Anim Hosp Assoc 37: 319-324.
14. Sueda K, Hart B L, Cliff K D (2008) Characterisation of plant eating in dogs. Appl Anim Behav Sci 111: 120-132.
15. Tynes V V, Hart B L, Bain M J (2007) Human-directed aggression in miniature pet pigs. J Am Vet Med Assoc 230: 385-389.
16. Gosling S D, Vazire S, Srivastava S, John O P (2004) Should we trust web-based studies? A comparative analysis of six preconceptions about internet questionnaires. Am Psychol 59: 93-104.
17. Reips U (2002) Standards for internet-based experimenting. Exp Psychol 49: 243-256.
18. Rhodes S D, Bowie D A, Hergenrather K C (2003) Collecting behavioral data using the world wide web: Considerations for researchers. J Epidemiol Comm Health 57: 68-73.
19. Bowman D D (2014) Georgis' Parasitology for Veterinarians, 10th ed. Elsevier Saunders, St. Louis.
20. Harrington F H, Asa C S (2003) Wolf Communication, in L D Mech, L Boitani (Eds.), Wolves: Behavior, Ecology, and Conservation. University of Chicago Press, Chicago. Page 101.
21. McKeown D, Luescher A, Machum M (1988) Coprophagia: Food for thought. Can Vet J 28: 849-850.

### Example 2

### Illustrative Method for Deterring Canine Coprophagy

The treatment should be prescribed and usually dispensed through a veterinarian who would explain the proper procedure for successfully conducting the treatment and list the possible problems.

To begin the treatment, the dog owner is instructed to maintain the stool-eating dog inside, in the morning, after an initial defecation until the available stools are treated. From this point on, all stools must be LiCl-treated as described below or picked up. If other dogs are around their stools should be LiCl-treated or picked up. All stools available to the problem dog, especially the fresh stools, must be LiCl-treated. The LiCl-treatment of all available fresh stools can be with a plastic-tip syringe containing a solution of 2 g LiCl in water or a gel. The syringe can hold a volume in the range of 2-3 ml containing 2 g LiCl so that about 0.5 ml of the solution is injected into portions of the stool, e.g., about 1-inch apart. The treatment can involve one syringe per stool to be treated. In order that the canine does not detect the LiCl on the stool, the LiCl composition is injected or impregnated below the surface of the stool such that it is not evident to the dog on the surface. For small-dog stools less of the LiCl composition in the syringe amount is used.

On the day of treatment the owner must be vigilant to treat all fresh stools. The owner should then observe the dog for which treatment is arranged. If the dog eats a treated stool, the owner should watch the dog after eating a treated stool. Some signs of nausea are expected along with vomiting. These are signs that the treatment is producing aversion. If the dog does not eat a treated stool the treatment procedure should be continued until the dog eats a treated stool. Once signs of nausea and coprophagia aversion are seen, then treatment can be stopped, and the dog should be observed for any eating of a stool. If the dog does eat another stool, the treatment program should be conducted again. If the dog shows prolonged severe signs of illness after eating a treated stool the veterinarian prescribing the treatment should be consulted.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of deterring a canine from ingesting feces, the method comprising:
a) introducing into the interior of mammalian feces a composition comprising a sufficient amount of lithium chloride (LiCl) to induce nausea in the canine upon or after ingestion of the feces;
b) orally administering or feeding the canine the LiCl-treated feces, thereby inducing nausea in the canine upon or after ingestion of the feces, wherein the nausea associated with the feces deters the canine from ingesting feces.

2. The method of claim 1, wherein the composition comprising LiCl comprises a concentration in the range of about 0.25 g/ml to about 2.0 g/ml LiCl.

3. The method of claim 1, wherein the composition comprising LiCl comprises an aqueous solution or gel.

4. The method of claim 1, wherein about 0.5 g to about 3.0 g of LiCl is introduced into about 50 g of feces.

5. The method of claim 1, wherein the composition comprising LiCl is not evident to the canine on the surface of the feces.

6. The method of claim 1, wherein the LiCl-treated feces comprises canine feces, feline feces, or equine feces.

7. The method of claim 6, wherein the LiCl-treated feces is the canine's own feces.

8. The method of claim 1, wherein the LiCl-treated feces was expelled within about 96 hours or the feces was frozen within about 96 hours after expulsion.

9. The method of claim 1, comprising performing steps a) and b) until ingestion of feces by the canine is reduced or inhibited.

10. The method of claim 1, wherein the canine is, in whole or in part, from a hound group breed, a terrier group breed, a herding group breed, a sporting group breed, a working group breed, or a toy group breed, as defined by the American Kennel Club (AKC).

11. A composition comprising mammalian feces comprising in the interior of the feces lithium chloride (LiCl) in an amount sufficient to induce nausea in a canine upon or after ingestion of the feces.

12. The composition of claim 11, wherein the feces comprises canine feces, feline feces, or equine feces.

13. The composition of claim 11, wherein the composition comprises from about 0.5 g to about 3.0 g of LiCl in the interior of about 50 g of feces.

14. The composition of claim 11, wherein the composition comprises substantially no LiCl on the surface of the feces.

15. The composition of claim 11, wherein the feces was expelled within about 96 hours or the feces was frozen within about 96 hours after expulsion.

16. The composition of claim 11, wherein the feces has been irradiated.

17. A syringe comprising a composition comprising LiCl at a concentration in the range of about 0.25 g/ml to about 2.0 g/ml LiCl.

18. The syringe of claim 17, wherein the syringe does not comprise a needle.

19. A kit, comprising:
the syringe of claim 17; and
instructions for using the syringe to introduce the composition into the interior of mammalian feces.
